SEQUENCE LISTING (3 sequences)

SEQ ID NO: 1 - DNA, 38 bp, Artificial Sequence
  aptamer
ccgcttttttttttttttttttttttttttttttgcgg

SEQ ID NO: 2 - DNA, 16 bp, Artificial Sequence
  DNAzyme
gggtgggagggtcggg

SEQ ID NO: 3 - DNA, 60 bp, Artificial Sequence
  nucleic acid element
tgggtgggagggtcgggccctcccgcttttttttttttttttttttttttttttttgcgg

(19) United States
(12) Patent Application Publication
(10) Pub. No.: US 2016/0202154 A1
(43) Pub. Date: Jul. 14, 2016

(54) METHOD FOR PRODUCING SAMPLE AND METHOD FOR ANALYZING TARGET

(71) Applicant: NEC SOLUTION INNOVATORS, LTD., Tokyo (JP)

(72) Inventors: Katsunori HORII, Tokyo (JP); Jou AKITOMI, Tokyo (JP); Naoto KANEKO, Tokyo (JP); Iwao WAGA, Tokyo (JP)

(73) Assignee: NEC Solution Innovators, Ltd., Tokyo (JP)

(21) Appl. No.: 14/916,942

(22) PCT Filed: Jun. 27, 2014
(86) PCT No.: PCT/JP2014/067118

§ 371 Date: Mar. 4, 2016

(30) Foreign Application Priority Data
Sep. 5, 2013 (JP) 2013-183857

(51) Int. Cl. G01N 1/30; G01N 33/538; G01N 33/04; G01N 33/53

(52) U.S. Cl. CPC G01N 1/30; G01N 33/5308; G01N 33/538; G01N 33/04

(57) ABSTRACT

The present invention is intended to provide a novel sensor for target analysis and a target analysis method using the same.

The sensor for target analysis according to the present invention includes a single-stranded nucleic acid molecule. The single-stranded nucleic acid molecule includes a first catalytic nucleic acid region (D1), a second catalytic nucleic acid region (D2), and a binding nucleic acid region (Ap) that binds to a target. The single-stranded nucleic acid molecule includes the first catalytic nucleic acid region (D1) at one end of the binding nucleic acid region (Ap) and the second catalytic nucleic acid region (D2) at the other end of the binding nucleic acid (Ap). In the absence of a target, the catalytic function by the first catalytic nucleic acid region (D1) and the second catalytic nucleic acid region (D2) is inhibited. In the presence of a target, the catalytic function is generated owing to formation of a G-quartet of the first catalytic nucleic acid region (D1) and the second catalytic nucleic acid region (D2) due to the contact between the target and the binding nucleic acid region (Ap).

### TECHNICAL FIELD

The present invention relates to a method for producing a sample and a method for analyzing a target.

### BACKGROUND ART

There is a demand for detection of a target in various fields such as a clinical treatment field, a food field, and an environment field, and the interaction with the target is commonly utilized for the detection. Commonly, a target is detected as follows by using a first binding substance that binds to the target and a second binding substance that binds to the first binding substance and is labeled with a labeling substance, for example. First, the first binding substance is caused to bind to the target in a sample, and then the labeled second binding substance is caused to bind to the first binding substance that is bound to the target to form a complex of the target, the first binding substance, and the labeled second binding substance. Then, by detecting the labeling substance of the labeled second binding substance in the complex, the target in the sample can be detected indirectly.

Commonly, antibodies are used as the first binding substance and the second binding substance, and oxidoreductase such as peroxidase is used as the labeling substance. However, in these years, a method utilizing a nucleic acid molecule that binds to a target and a nucleic acid molecule that has a catalytic function similar to an enzyme as new tools in place of antibodies and an enzyme is proposed. The former nucleic acid molecule (binding nucleic acid molecule) is a so-called aptamer and the latter nucleic acid molecule (catalytic nucleic acid molecule) is a so-called DNAzyme, RNAzyme, or the like. Such nucleic acid molecules can be utilized for detection of the target as a nucleic acid element in which the binding nucleic acid molecule and the catalytic nucleic acid molecule are linked. For example, such nucleic acid molecules allow simpler analyses and smaller analysis devices, for example.

### CITATION LIST

### Non-Patent Document(s)

Non-Patent Document 1: Teller et al., Anal. Chem., 2009, vol. 81, pp. 9114-9119

### SUMMARY OF INVENTION

### Problem to be Solved by the Invention

However, detection of a target using the catalytic nucleic acid molecule is difficult depending on a specimen for the following reasons. Among the specimens, for example, milk such as cow's milk and milk products such as milk powder and the like contain protein, lipid, and inhibitors that inhibit the catalytic function of the catalytic nucleic acid molecule as contaminants. Therefore, for performing the detection method using the catalytic nucleic acid molecule, it is required to remove contaminants from the specimen by applying a pretreatment thereto to prepare a sample to be subjected to an analysis. For example, a coagulation treatment using an organic solvent is required for removing contaminants such as protein, lipid, and the like. However, the inventors of the present invention found that organic solvents sometimes mixed into samples prepared using organic solvents and that the catalytic nucleic acid molecules do not work because of the organic solvents mixed in the samples. Therefore, it was found that it is important to apply a pretreatment to a specimen without requiring an organic solvent to prepare a sample in the detection of a target using the catalytic nucleic acid molecule.

Hence, the present invention is intended to provide a method for producing a sample to be subjected to a target analysis using the catalytic nucleic acid molecule without requiring an organic solvent and a method for analyzing a target using the sample.

### Means for Solving Problem

The present invention provides a method for producing a sample including: bringing a specimen into contact with a cationic polymer in an aqueous mixture containing the specimen and the cationic polymer; recovering a liquid fraction containing a target in the specimen from the aqueous mixture by solid-liquid separation; and recovering a sample containing the target from the liquid fraction by column chromatography using an aqueous solvent, wherein the sample is a sample to be subjected to a method for analyzing a target using a catalytic nucleic acid molecule that generates a catalytic function.

The present invention also provides a method for analyzing a target including: bringing the sample produced by the method according to the present invention into contact with a first binding substance that binds to a target and a catalytic nucleic acid molecule that generates a catalytic function to form a complex of the target in the sample, the first binding substance, and the catalytic nucleic acid molecule; and detecting the catalytic function of the catalytic nucleic acid molecule in the complex to detect the target in the sample.

### Effects of the Invention

According to the present invention, a sample to be subjected to a method for analyzing a target using the catalytic nucleic acid molecule can be produced, without substantially requiring an organic solvent, by a coagulation treatment using a cationic polymer in an aqueous mixture, column chromatography using an aqueous solvent, and the like. Since a sample prepared according to the present invention contains substantially no organic solvent, as described above, the influence on the function of the catalytic nucleic acid molecule due to the organic solvent can be suppressed. Therefore, for example, the present invention is very useful for researches and tests in various fields such as a clinical treatment field, a food field, and an environment field.

### BRIEF DESCRIPTION OF DRAWINGS

FIG. 1 is a graph showing the luminescent intensity of a reaction solution of melamine analysis using a nucleic acid element in Example 1 of the present invention.

FIG. 2 is a graph showing the elution pattern of melamine by cation exchange chromatography in Example 2 of the present invention.

FIG. 3 is a graph showing the result of the luminescent intensity indicating the detection of melamine in Example 3 of the present invention.

### DESCRIPTION OF EXEMPLARY EMBODIMENT

(Production Method of Sample)

As described above, the production method of a sample according to the present invention is characterized in that it includes: bringing a specimen into contact with a cationic polymer in an aqueous mixture containing the specimen and the cationic polymer; recovering a liquid fraction containing a target in the specimen from the aqueous mixture by solid-liquid separation; and recovering a sample containing the target from the liquid fraction by column chromatography using an aqueous solvent, wherein the sample is a sample to be subjected to a method for analyzing a target using a catalytic nucleic acid molecule that generates a catalytic function.

The production method of a sample according to the present invention can also be referred to as a preparation method of a sample or a pretreatment method of a specimen, for example. In the production method of a sample according to the present invention, the contact step, the liquid fraction recovery step, and the sample recovery step can also be referred to as a pretreatment step of a specimen, for example.

In the present invention, a specimen to be pretreated can be either a liquid specimen or a solid specimen, for example. The type of the specimen is not particularly limited, and examples thereof include food specimens, biological specimens, and environmental specimens. The food may be liquid food such as beverages or solid food, and examples thereof include milk such as cow's milk, milk products (e.g., dried milk, milk powder, etc.) such as cow's milk products, raw milk, and processed milk. Examples of the biological specimen include blood, urine, and saliva. Examples of the environmental specimen include seawater, river water, pond water, wastewater such as sewage and industrial wastewater, sludge, and soil.

In the present invention, the target is not particularly limited and the target can be any substance. Examples of the target include low-molecular compounds, microorganisms, viruses, food allergens, agricultural chemicals, and mycotoxin. Specifically, the target can be melamine or the like.

The contact step is, as described above, a step of bringing the specimen into contact with the cationic polymer in the aqueous mixture containing the specimen and the cationic polymer.

The cationic polymer can be any polymer as long as it is cationic and the type of the cationic polymer is not particularly limited. The cationic polymer preferably has the following chemical property, for example. The number average molecular weight (Mn) of the cationic polymer is, for example, from 50 to 2000, from 100 to 1000, or from 150 to 250.

The cationic polymer is not particularly limited and is preferably dimethylaminoethyl methacrylate methylchloride salt homopolymer represented by the chemical formula (1), polydimethyldiallylammonium chloride represented by the chemical formula (2), or the like, for example. In the chemical formulae, the degree of polymerization (n) is not particularly limited.

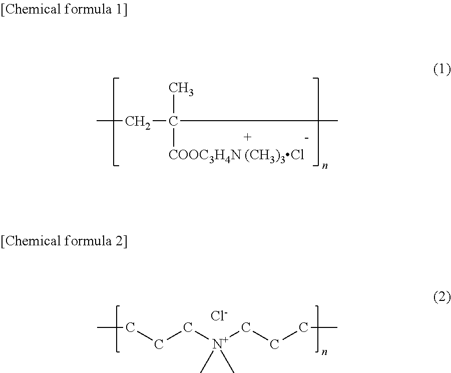

The polymer represented by the chemical formula (1) may be obtained by synthesis or by purchase of a commercially available polymer, for example. For example, commercially available cationic agents called Tai Polymer TC-580, TC-580L, TC-580H, TC-580FL, TC-580VL, TC-5805, TC-570, and TC-560 (TAIMEI CHEMICALS Co., Ltd.) can be used.

The number average molecular weight (Mn) of the polymer represented by the chemical formula (2) is, for example, from 50 to 2000, from 100 to 1000, or from 150 to 250.

The polymer represented by the chemical formula (2) may be obtained by synthesis or by purchase of a commercially available polymer, for example. For example, commercially available cationic agents called Tai Polymer TC-7400, TC-7100, TC-7200, and TC-7500 (TAIMEI CHEMICALS Co., Ltd.) can be used.

One of the cationic polymers may be used alone or two or more of them may be used in combination, for example. As a specific example, each of the polymer represented by the chemical formula (1) and the polymer represented by the chemical formula (2) may be used alone or both of them may be used in combination. When both of them are used in combination, the volume ratio between the polymer represented by the chemical formula (1) and the polymer represented by the chemical formula (2) is, for example, 1:0.01 to 0.1 or 1:0.01 to 0.03.

It is preferred that the aqueous mixture used in the contact step contains substantially no organic solvent, and it is particularly preferred that the aqueous mixture is consisting of an aqueous solvent, for example. “The aqueous mixture contains substantially no organic solvent” means that, even when a finally obtained sample contains an organic solvent, the amount of the organic solvent is in the range that does not affect the function of the catalytic nucleic acid molecule when the sample is subjected to an analysis method of a target using the catalytic nucleic acid molecule, for example. In the case where the aqueous mixture contains an organic solvent, the content ratio of the organic solvent is, for example, 50 vol % or less, 30 vol % or less, 10 vol % or less, or the detection limit or less. “The detection limit or less” means an undetectable threshold or less in the detection of an organic solvent using HPLC or the like, for example.

The aqueous mixture may be prepared by mixing the specimen and the cationic polymer or by mixing the specimen, the cationic polymer, and a dispersion medium, for example. The dispersion medium is, for example, an aqueous solvent.

The aqueous solvent is not particularly limited, and examples thereof include water and buffer solutions. Examples of the buffer solution include MES (2-(N-morpholino)ethanesulfonic acid), Tris, MOPS, HEPES, and TES. The pH of the buffer solution is not particularly limited and is, for example, from 5 to 12 or from 5 to 9.

In the case where the specimen, the cationic polymer, and the aqueous solvent (dispersion medium) are mixed in the preparation of the aqueous mixture, the order of mixing them is not particularly limited. For example, three of them may be mixed at the same time or two of them may be mixed first and then the rest of them may be mixed therein. As a specific example of the latter case, for example, the specimen and the aqueous solvent may be mixed first and then the cationic polymer may be mixed therein; the cationic polymer and the aqueous solvent may be mixed first and then the specimen may be mixed therein; or the specimen and the cationic polymer may be mixed first and then the aqueous solvent may be mixed therein. In terms of handleability, when the specimen is solid, it is preferred that the solid specimen is dispersed in the aqueous solvent and then mixed with the cationic polymer, for example. It is also preferred that the cationic polymer is preliminarily dispersed in the aqueous solvent and then mixed with the specimen, for example.

The proportion of the specimen in the aqueous mixture is not particularly limited. The volume ratio between the specimen (S) and the cationic polymer (P) in the mixture is not particularly limited.

The conditions for the contact between the specimen and the cationic polymer in the aqueous mixture are not particularly limited. The temperature is, for example, from 4° C. to 60° C. or from 4° C. to 37° C. and the time is, for example, from 10 seconds to 60 minutes or from 30 seconds to 5 minutes. It is preferred that the components of the aqueous mixture are mixed by stirring and then the resultant is caused to stand still, for example. The time for stirring is, for example, from 10 seconds to 10 minutes and the time for standing still is, for example, from 10 to 60 minutes.

The liquid fraction recovery step is, as described above, a step of recovering a liquid fraction containing a target in the specimen from the aqueous mixture by solid-liquid separation.

The method for the solid-liquid separation is not particularly limited. The solid-liquid separation may be performed by causing the aqueous mixture to stand still, by filtering the aqueous mixture, or by performing centrifugal separation of the aqueous mixture, for example.

The sample recovery step is a step of recovering a sample containing the target from the liquid fraction by column chromatography using an aqueous solvent. The sample recovery by the column chromatography is characterized in that it uses the aqueous solvent, and other conditions are not particularly limited.

The type of the column chromatography is not particularly limited and can be determined according to the type of a target, for example. The sample recovery may be performed, for example, as follows. That is, an adsorption fraction containing the target may be recovered by causing the column to adsorb a target and eluting the target. Also a non-adsorption fraction containing the target may be recovered by causing the column to adsorb components except for the target.

A solid phase extraction column is preferably used in the column chromatography owing to its superior handleability, for example.

Examples of the column chromatography include cation exchange column chromatography and anion exchange column chromatography. The cation exchange group of the former chromatography is not particularly limited, and examples thereof include 2-carboxyethyl group (—CH₂CH₂—COOH) and 2-(4-sulfophenyl) ethyl group (—CH₂CH₂—C₆H₄—SO₃H). As specific examples, commercially available products such as Strata WCX (product name, Phenomenex Inc.), Strata SCX (product name, Phenomenex Inc.), and the like can be used. The anion exchange group of the latter chromatography is not particularly limited, and examples thereof include 3-(trimethylammonium) propyl group (—CH₂CH₂—CH₂—N(CH₃)₃) and 4-amino propyl group (—CH₂CH₂—CH₂—NH₂). As specific examples, commercially available products such as Strata NH₂/WAX (product name, Phenomenex Inc.), Strata SX (Phenomenex Inc.), and the like can be used.

As described above, the column chromatography can be determined according to the type of the target. The column chromatography to a specific target is described below. However, the present invention is not limited to these examples.

In the case where the target is melamine, for example, it is preferred that the adsorption fraction is recovered as a sample by cation exchange column chromatography. The cation exchange group of the cation exchange chromatography is preferably a 2-carboxyethyl group (—CH₂CH₂—COOH) or a 2-(4-sulfophenyl) ethyl group (—CH₂CH₂—C₆H₄—SO₃H), for example.

In the case where a sample containing melamine is recovered by the cation exchange column chromatography, for example, application of the liquid fraction, washing of the column, and elution of the adsorption fraction containing melamine can be performed under the following conditions.

### (1) Application

Concentration of buffer solution: 50 mmol/L

Type of buffer solution: MES

pH of buffer solution: 5.5 to 6.5

### (2) Washing

Concentration of buffer solution: 50 mmol/L

Type of buffer solution: MES

pH of buffer solution: 5.5 to 6.5

### (3) Elution

Concentration of buffer solution: 100 mmol/L

Type of buffer solution: HEPES

pH of buffer solution: 7 to 8

A sample obtained in this manner can be used as a sample to be subjected to an analysis method of a target using the catalytic nucleic acid molecule as described above.

The catalytic nucleic acid molecule is not particularly limited, and examples thereof include DNAzyme and RNAzyme. Specifically, the description for the analysis method that will be described below can be applied.

(Analysis Method of Target)

As described above, the analysis method of a target according to the present invention is characterized in that it includes: bringing the sample produced by the method according to the present invention into contact with a first binding substance that binds to a target and a catalytic nucleic acid molecule that generates a catalytic function to form a complex of the target in the sample, the first binding substance, and the catalytic nucleic acid molecule; and detecting the catalytic function of the catalytic nucleic acid molecule in the complex to detect the target in the sample.

The present invention is characterized in that a sample produced by the production method according to the present invention is subjected to the analysis method using the catalytic nucleic acid molecule, and other steps and conditions are not particularly limited.

The first binding substance that binds to the target is not particularly limited, and examples thereof include the binding nucleic acid molecule and the antibody. Among them, the binding nucleic acid molecule is preferable. The binding nucleic acid molecule can also be referred to as an aptamer.

In the complex forming step, the first binding substance and the catalytic nucleic acid molecule may be used as an analysis element in which each of them are preliminarily linked or may be used separately, for example. Hereinafter, the embodiment in which the analysis element is used is referred to as a first embodiment and the embodiment in which the first binding substance and the catalytic nucleic acid molecule are used separately is referred to as a second embodiment. The present invention is not limited to these embodiments.

The first embodiment is an embodiment that uses an analysis element in which the first binding substance and the catalytic nucleic acid molecule are preliminarily linked.

In the first embodiment, the first binding substance can be, for example, either the binding nucleic acid molecule or the antibody and is preferably the binding nucleic acid molecule. The analysis element is preferably a nucleic acid element for use in analysis in which the binding nucleic acid molecule and the catalytic nucleic acid molecule are linked, for example. The form of linkage between the binding nucleic acid molecule and the catalytic nucleic acid molecule is not particularly limited, and the analysis element may be a single-stranded nucleic acid molecule or a double-stranded nucleic acid molecule, for example.

In the first embodiment, for example, a target in the sample and the first binding substance of the analysis element are caused to bind to each other by bringing the sample into contact with the analysis element to form a complex of the target and the analysis element (the first binding substance and the catalytic nucleic acid molecule). Then, by detecting the catalytic function of the catalytic nucleic acid molecule in the complex, the target can be detected indirectly. The first embodiment may further include a step of removing the analysis element not involved in the formation of the complex between the complex forming step and the detection step.

The second embodiment is an embodiment that uses the first binding substance and the catalytic nucleic acid molecule separately.

In the second embodiment, the first binding substance can be, for example, either the binding nucleic acid molecule or the antibody and is preferably the binding nucleic acid molecule. In the second embodiment, the catalytic nucleic acid molecule is preferably modified with a second binding substance that binds to the first binding substance, for example. Specifically, it is preferred that the first binding substance and a second binding substance that is modified with the catalytic nucleic acid molecule and binds to the first binding substance are brought into contact with the sample separately. The second binding substance can be any substance as long as it is bindable to the first binding substance that binds to the target, and is preferably a substance different from the target. Furthermore, the second binding substance can be, for example, either a binding nucleic acid molecule that binds to the first binding substance or an antibody that binds to the first binding substance, and is preferably the binding nucleic acid molecule.

In the second embodiment, for example, a target in the sample and the first binding substance are caused to bind to each other by bringing the sample into contact with the first binding substance and the modified second binding molecule modified with the catalytic nucleic acid molecule, and then a complex of the target, the first binding substance, and the second binding substance is formed by binding the modified second binding substance to the first binding substance. On this occasion, the order of contact with the sample is not particularly limited. The first binding substance and the modified second binding substance may be brought into contact with the sample at the same time, the first binding substance may be brought into contact with the sample first and then the modified second binding substance may be brought into contact with them, the modified second binding substance may be brought into contact with the sample first and then the first binding substance may be brought into contact with them, or the first binding substance may be brought into contact with the modified second binding substance first and then they may be brought into contact with the sample.

Then, by detecting the catalytic function of the catalytic nucleic acid molecule of the modified second binding substance in the complex, the target can be detected indirectly. The second embodiment may further include a step of removing the first binding substance and the modified second binding substance not involved in the formation of the complex between the complex forming step and the detection step.

In the present invention, the catalytic nucleic acid molecule can be any nucleic acid molecule as long as it generates a catalytic function. The catalytic function is not particularly limited and is, for example, the catalytic function of a redox reaction. The redox reaction can be a reaction in which electrons are transferred between two substrates in a course of producing a product from the substrates, for example. The type of the redox reaction is not particularly limited. The catalytic function of the redox reaction can be, for example, an activity similar to an enzyme and specifically is, for example, an activity similar to peroxidase (hereinafter referred to as “peroxidase-like activity”). The peroxidase activity can be, for example, horseradish peroxidase (HRP) activity. In the case where the catalytic nucleic acid molecule is a DNA sequence, it can be called DNA enzyme or DNAzyme. In the case where the catalytic nucleic acid molecule is an RNA sequence, it can be called RNA enzyme or RNAzyme.

The catalytic nucleic acid molecule is preferably a nucleic acid sequence that forms a G-quartet (also referred to as G-tetrad) and is more preferably a nucleic acid sequence that forms a guanine quadruplex (also referred to as G-quadruplex). The G-tetrad is, for example, a planar structure formed of four guanine bases. The G-quadruplex is, for example, a structure in which G-tetrads are stacked on top of each other. The G-tetrad and the G-quadruplex are formed in a nucleic acid that repeatedly includes G-rich structural motifs, for example. The G-tetrad can be either a parallel type or an antiparallel type, for example. It is preferred that the G-tetrad is a parallel type.

The catalytic nucleic acid molecule is preferably a nucleic acid sequence that is bindable to porphyrin. Specifically, the catalytic nucleic acid molecule is preferably a nucleic acid sequence that forms a G-tetrad and is bindable to porphyrin. The nucleic acid sequence including a G-tetrad is known to generate the catalytic function of the redox reaction by binding to porphyrin to form a complex, for example.

The porphyrin is not particularly limited, and examples thereof include unsubstituted porphyrin and the derivatives thereof. Examples of the derivative include substituted porphyrin and metal porphyrin which is a complex of porphyrin and a metal element. The substituted porphyrin can be, for example, N-Methylmesoporphyrin and the like. The metal porphyrin can be, for example, hemin, which is a ferric complex, and the like. The porphyrin is preferably the metal porphyrin and is more preferably hemin, for example.

The sequence of the catalytic nucleic acid molecule is not particularly limited and can be any sequence. Specifically, for example, the sequence of a well-known catalytic nucleic acid molecule that generates a catalytic function and the partial sequence of such a catalytic nucleic acid molecule can be employed as the sequence of the catalytic nucleic acid molecule. Examples of the catalytic nucleic acid molecule having a peroxidase activity include DNAzyme disclosed in the following articles (1) to (4):

(1) Travascio et al., Chem. Biol., 1998, vol. 5, pp. 505-517;

### (2) Cheng et al., Biochemistry, 2009, vol. 48, pp. 7817-7823;

(3) Teller et al., Anal. Chem., 2009, vol. 81, pp. 9114-9119; and
(4) Tao et al., Anal. Chem., 2009, vol. 81, pp. 2144-2149.

The binding substance that binds to the target can be selected according to a target, for example. As a specific example, in the case where the target is melamine, the binding substance can be, for example, the binding nucleic acid molecule of the sequence disclosed in the following article:

### Aihui Liang et al., J. Fluoresc., 2011, vol. 21, pp. 1907-1912.

Examples of the building block of the catalytic nucleic acid molecule include ribonucleotide residues, deoxyribonucleotide residues, and nucleotide residues of the derivatives thereof. Furthermore, the catalytic nucleic acid molecule may include a non-nucleotide residue such as peptide nucleic acid (PNA), locked nucleic acid (LNA), or the like.

The method for detecting the catalytic function of the catalytic nucleic acid molecule is not particularly limited and can be determined appropriately according to the catalytic function. For example, it is preferred that the signal generated by the catalytic function is measured. The signal is not particularly limited, and examples thereof include optical signals and electrochemical signals. Examples of the optical signal include color development signals, luminescent signals, and fluorescent signals.

It is preferred that the signal is generated from a substrate by the catalytic function of the catalytic nucleic acid molecule, for example. Hence, it is preferred that the detection of the catalytic function is performed in the presence of a substrate appropriate to the catalytic function of the catalytic nucleic acid molecule, for example.

Examples of the substrate include a substrate that produces a color development product, a luminescent product, or a fluorescent product by the catalytic function; a color development, luminescent, or fluorescent substrate that produces a product quenching its color development, luminescence, or fluorescence by the catalytic function; and a substrate that produces a product changing its color development, luminescence, or fluorescence by the catalytic function. Such substrates allow detection of the catalytic function by visually examining the presence or absence of the color development, the luminescence, or the fluorescence or the change, the intensity, or the like of the color development, the luminescence, or the fluorescence as a signal, for example. Furthermore, for example, by measuring the absorbance, the reflectance, the fluorescent intensity, or the like as a signal by an optical method, the catalytic function can be detected. The catalytic function can be, for example, the catalytic function of the redox reaction as mentioned above.

In the case where the catalytic nucleic acid molecule has the catalytic function of the redox reaction, for example, the substrate can be a substrate that can give and receive electrons. In this case, a product is produced from the substrate by the catalytic nucleic acid molecule, for example, and electrons are transferred in a course of producing a product from the substrate. The transfer of electrons can be electrochemically detected as an electrical signal by applying a voltage to an electrode, for example. The detection of the electrical signal can be performed by measuring the intensity of the electrical signal such as a current or the like, for example.

The substrate is not particularly limited and examples thereof include hydrogen peroxide, 3,3′,5,5′-Tetramethylbenzidine (TMB), 1,2-Phenylenediamine (OPD), 2,2′-Azinobis(3-ethylbenzothiazoline-6-sulfonic Acid Ammonium Salt (ABTS), 3,3′-Diaminobenzidine (DAB), 3,3′-Diaminobenzidine Tetrahydrochloride Hydrate (DAB4HCl), 3-Amino-9-ethylcarbazole (AEC), 4-Chloro-1-naphthol (4C1N), 2,4,6-Tribromo-3-hydroxybenzoic Acid, 2,4-Dichlorophenol, 4-Aminoantipyrine, 4-Aminoantipyrine Hydrochloride, and luminol.

The detection conditions of the catalytic function are not particularly limited, and the temperature is, for example, from 15° C. to 37° C. and the time is, for example, from 10 seconds to 900 seconds.

In the detection of the catalytic function, porphyrin may be caused to coexist besides the substrate, for example. Some known DNAzymes show higher redox activities by forming complexes with porphyrin, for example. Hence, for example, the redox activity can be detected by causing porphyrin to coexist to form a complex of the catalytic nucleic acid molecule and the porphyrin.

The porphyrin is not particularly limited, and examples thereof include unsubstituted porphyrin and the derivatives thereof. Examples of the derivative include substituted porphyrin and metal porphyrin which is a complex of porphyrin and a metal element. The substituted porphyrin can be, for example, N-Methylmesoporphyrin and the like. The metal porphyrin can be, for example, hemin, which is a ferric complex, and the like. The porphyrin is preferably the metal porphyrin and is more preferably hemin, for example.

Hereinafter, the present invention will be described in detail with reference to examples. It is to be noted, however, that the present invention is not limited thereto.

### EXAMPLES

### Example 1

A specimen containing melamine was pretreated to prepare a sample, and melamine in the sample was analyzed.

(1) Preparation of Specimen

5.2 g of commercially available dried milk (product name: Hagukumi (dried milk), MORINAGA MILK INDUSTRY CO., LTD.) was suspended in 40 mL of water to prepare a dried milk solution. Then melamine was added to the dried milk solution to achieve a concentration of 15 mol/L to prepare a dried milk solution containing melamine. Also melamine was added to commercially available milk (raw milk: 100%, product name: Meiji Oishii Gyunyu, Meiji Holdings Co., Ltd.) to achieve a concentration of 15 mol/L to prepare milk containing melamine. A dried milk solution containing no melamine, a dried milk solution containing melamine, milk containing no melamine, and milk containing melamine were used as specimens.

(2) Preparation of Sample

Distilled water was mixed with 10 mL of each specimen in equal amount, then a cationic polymer (product name: TC-7400, TAIMEI CHEMICALS Co., Ltd.) was mixed therewith so as to achieve the final concentration of 1%, and the resultant was stirred for 10 seconds. Subsequently, a cationic polymer (product name: TC-580, TAIMEI CHEMICALS Co., Ltd.) was mixed with the mixture so as to achieve the final concentration of 0.03%, and the resultant was stirred for 10 seconds to prepare an aqueous mixture. The aqueous mixture was caused to stand still for 5 minutes, and then the resultant was subjected to centrifugal separation (15,000 rpm, 15 minutes) to recover a liquid fraction. The liquid fraction was again subjected to the centrifugal separation under the same conditions to recover a liquid fraction.

The liquid fraction was subjected to cation exchange column chromatography under the following conditions to recover an adsorption fraction. This adsorption fraction was used as a sample.

Ion-exchange resin: Strata WCX (product name, Phenomenex Inc.)
Column size: diameter 0.9 cm×length 6.5 cm
Applied liquid fraction: 3 mL
Buffer solution for equilibration: 3 mmol/L MES buffer (pH 5.5)
Buffer solution for washing: 50 mmol/L Tris-HCl buffer (pH 7.4)
Buffer solution for elution: 100 mmol/L Tris-HCl buffer (pH 7.4)
Temperature: 25° C. (room temperature)

(3) Chemiluminescent Analysis

Subsequently, the melamine concentration of the sample was measured by using a nucleic acid element in which an aptamer that binds to melamine and DNAzyme are linked.

As the nucleic acid element, a single-stranded nucleic acid element (SEQ ID NO: 3) including a melamine aptamer (SEQ ID NO: 1) as a binding nucleic acid molecule that binds to melamine and DNAzyme neco0584 (SEQ ID NO: 2) as a catalytic nucleic acid molecule was used. In the sequence of the nucleic acid element, the underlined part at the 5′ side is DNAzyme and the underlined part at the 3′ side is a melamine aptamer.

Melamine aptamer
(SEQ ID NO: 1)
CCGCTTTTTTTTTTTTTTTTTTTTTTTTTTTTTTGCGG
DNAzyme
(SEQ ID NO: 2)
GGGTGGGAGGGTCGGG
Nucleic acid element
(SEQ ID NO: 3)
TGGGTGGGAGGGTCGGGCCCTCCCGCTTTTTTTTTTTTTTTTTTTTTT
TTTTTTTTGCGG

1 mL of the sample, the following reagent 1, and the following reagent 2 were added to an Eppendorf tube in this order, the resultant was cause to react at 25° C. for 60 seconds, and then the relative chemiluminescent intensity (RLU) of the reaction solution was measured. The concentrations in the composition below were the final concentrations in the reaction solution (hereinafter, the same applies). For the measurement, a measurement apparatus (product name: TECAN infinite, TECAN) was used. As the substrate, L-012 (Wako Pure Chemical Industries, Ltd.), which is a luminol derivative, was used.

### (Reagent 1)

| | | |
|---|---|---|
| 250 | nmol/L | nucleic acid element |
| 125 | nmol/L | hemin |
| 50 | mmol/L | EDTA |
| 20 | mmol/L | KCl |

### (Reagent 2)

| | | |
|---|---|---|
| 25 | μmol/L | L-012 |
| 25 | μmol/L | H₂O₂ |

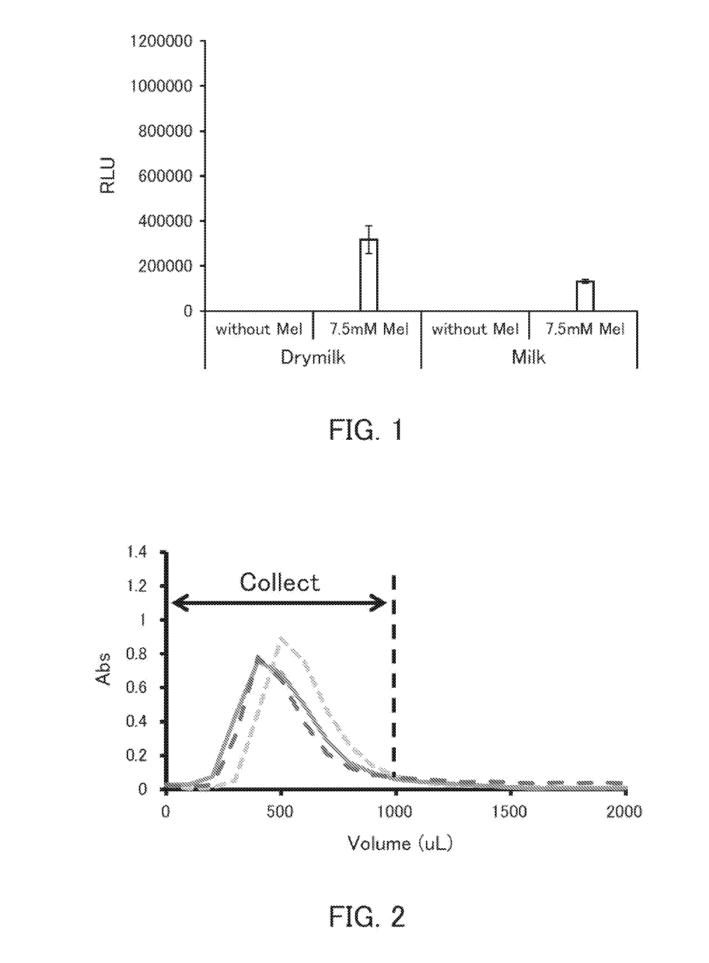

The results are shown in FIG. 1. FIG. 1 is a graph showing the luminescent intensity (RLU) of the reaction solution. As shown in FIG. 1, luminescence was not observed in a sample prepared from a specimen containing no melamine, whereas luminescence was observed in a sample prepared from a specimen containing melamine. From these results, it was found that contaminants such as protein, lipid, and the like could be removed from a specimen of milk or dried milk only with an aqueous solvent without using an organic solvent and a sample containing melamine could be recovered according to the method of the present invention. Furthermore, since an organic solvent was not used, inhibition of the function of DNAzyme due to an organic solvent was suppressed in the melamine analysis utilizing DNAzyme, and melamine could be detected.

### Example 2

A sample was recovered from milk containing melamine.

(1) Preparation of Specimen

Melamine was added to 5 mL of commercially available milk (raw milk: 100%, product name: Meiji Oishii Gyunyu, Meiji Holdings Co., Ltd.) so as to achieve a concentration of 4 mmol/L to prepare milk containing melamine (melamine final concentration: 4 mmol/L, milk final concentration: 100%). This milk containing melamine was used as a specimen.

(2) Preparation of Sample

1.3 mL of 10% (v/v) cationic polymer (product name: TC7400, TAIMEI CHEMICALS Co., Ltd.) was added to the whole specimen so as to achieve a polymer final concentration of 1% (v/v), the resultant was mixed for 10 seconds, 1.7 mL of 0.5% (v/v) cationic polymer (product name: TC-580, TAIMEI CHEMICALS Co., Ltd.) was added thereto so as to achieve a polymer final concentration of 0.03% (v/v), and the resultant was mixed for 10 seconds to prepare an aqueous mixture. The aqueous mixture was caused to stand still for 5 minutes, and then the resultant was subjected to centrifugal separation (15,000 rpm, 15 minutes) to recover a liquid fraction. The liquid fraction was again subjected to the centrifugal separation under the same conditions and 4 mL of liquid fraction was recovered.

210 μL of 1 mol/L MES buffer solution (pH 5.5) was added to 4 mL of the liquid fraction so as to achieve a final concentration of 50 mmol/L (melamine final concentration: 2 mmol/L, milk final concentration: 50%).

Then, the liquid fraction was subjected to cation exchange column chromatography under the following conditions.

Ion-exchange resin: Strata SCX (product name, Phenomenex Inc.)
Column size: diameter 0.9 cm×length 6.5 cm
Applied liquid fraction: 3 mL
Buffer solution for equilibration: 50 mmol/L MES buffer solution (pH 5.5) 3 mL
Buffer solution for washing:
First time: 50 mmol/L MES buffer solution (pH 5.5) 3 mL
Second time: 50 mmol/L Tris-HCl buffer solution (pH 7.4) 0.5 mL
Buffer solution for elution: 100 mmol/L Tris-HCl (pH 8.0)

### Temperature: 25° C.

With reference to the fraction recovered by the column (washed fraction and eluted fraction), the absorbance of melamine at the absorption wavelength of 248 nm was measured over time. The same treatment was performed three times with respect to the specimen. The results are shown in FIG. 2. FIG. 2 is a graph showing the elution pattern of melamine by the cation exchange chromatography. The vertical axis indicates the absorbance and the horizontal axis indicates the volume of the recovered fraction. As shown in FIG. 2, in all three treatments, melamine could be recovered with the total amount of solution of 1000 μL.

### Example 3

A sample was recovered from milk containing melamine and melamine was detected by chemiluminescent analysis using a nucleic acid element.

(1) Preparation of Specimen

Melamine was added to 5 mL of commercially available milk (raw milk: 100%, product name: Meiji Oishii Gyunyu, Meiji Holdings Co., Ltd.) so as to achieve a final concentration of 4 mmol/L to prepare milk containing melamine (melamine final concentration: 4 mmol/L, milk final concentration: 100%). This milk containing melamine was used as a specimen. Furthermore, milk containing no melamine was used as a control.

(2) Preparation of Sample

Melamine fraction was recovered in the same manner as in (2) of Example 2. The elution pattern of melamine by cation exchange chromatography was the same as that of Example 2 and melamine could be recovered with the total amount of solution of 1000 μL. 1000 μL of this recovered fraction was used as a sample.

(3) Chemiluminescent Analysis

Subsequently, the melamine concentration of the sample was measured by using the nucleic acid element in the same manner as in Example 1. Furthermore, as a Comparative Example, the chemiluminescent analysis was performed with respect to milk containing melamine and milk containing no melamine without being treated. Also, as a Comparative Example, the chemiluminescent analysis was performed with respect to the liquid fraction treated with the cationic polymer without subjecting to column chromatography.

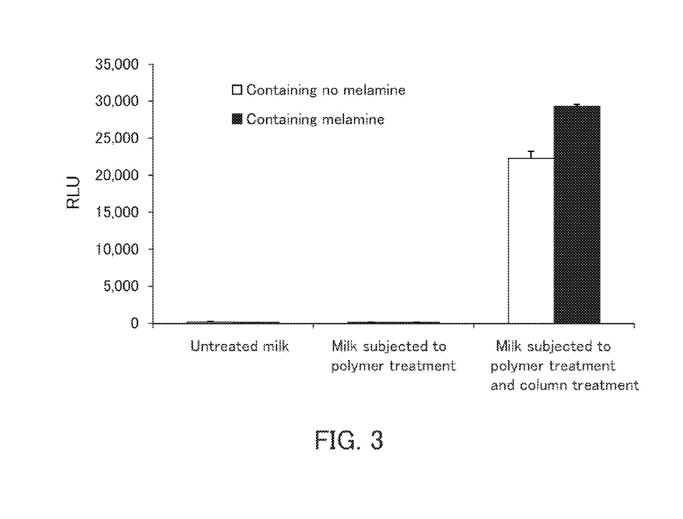

The results are shown in FIG. 3. FIG. 3 is a graph showing the luminescent intensity (RLU) of the reaction solution. As shown in FIG. 3, luminescence was not observed in milk containing melamine and milk containing no melamine in the case where they were not treated or they were subjected only to cationic polymer treatment, and milk containing melamine and milk containing no melamine could not be distinguished. In contrast, in the case where milk containing melamine and milk containing no melamine were subjected to the cationic polymer treatment and the column chromatography treatment, milk containing melamine showed significantly high luminescent intensity as compared to milk containing no melamine. From this result, it was found that melamine could be detected by recovering melamine from a specimen of milk or dried milk without using an organic solvent according to the method of the present invention.

While the present invention has been described above with reference to illustrative embodiments, the present invention is by no means limited thereto. Various changes that may become apparent to those skilled in the art may be made in the configuration and specifics of the present invention without departing from the scope of the present invention.

This application is based upon and claims the benefit of priority from Japanese patent application No. 2013-183857, filed on Sep. 5, 2013, the disclosure of which is incorporated herein its entirety by reference.

### INDUSTRIAL APPLICABILITY

According to the production method of a sample of the present invention, a sample to be subjected to a method for analyzing a target using the catalytic nucleic acid molecule can be produced, without substantially requiring an organic solvent, by a coagulation treatment using a cationic polymer in an aqueous mixture, column chromatography using an aqueous solvent, and the like. Since a sample prepared according to the present invention contains substantially no organic solvent, as described above, the influence on the function of the catalytic nucleic acid molecule due to the organic solvent can be suppressed. Therefore, for example, the present invention is very useful for researches and tests in various fields such as a clinical treatment field, a food field, and an environment field.

[Sequence Listing] 2014.04.28_TF14012WO_ST25.txt

1. A method for producing a sample, comprising:
bringing a specimen into contact with a cationic polymer in an aqueous mixture containing the specimen and the cationic polymer;
recovering a liquid fraction containing a target in the specimen from the aqueous mixture by solid-liquid separation; and
recovering a sample containing the target from the liquid fraction by column chromatography using an aqueous solvent, wherein
the sample is a sample to be subjected to a method for analyzing a target using a catalytic nucleic acid molecule that generates a catalytic function.

2. The method according to claim 1, wherein the specimen is a biological specimen.

3. The method according to claim 1, wherein the specimen is milk or a milk product.

4. The method according to claim 1, wherein the specimen is cow's milk or a cow's milk product.

5. The method according to claim 1, wherein the target is nonpeptide, non-protein, and non-lipid.

6. The method according to claim 1, wherein the target is melamine.

7. The method according to claim 1, wherein the aqueous mixture is a mixture containing the specimen, the cationic polymer, and an aqueous solvent.

8. The method according to claim 1, wherein the solid-liquid separation in the liquid fraction recovery step is centrifugal separation of the mixture.

9. The method according to claim 1, wherein a filler of the column chromatography is a cation exchange resin or an anion exchange resin.

10. The method according to claim 9, wherein the cation exchange resin is a resin including at least one of 2-carboxyethyl group (—CH₂CH₂—COOH) and 2-(4-sulfophenyl) ethyl group (—CH₂CH₂—C₆H₄—SO₃H).

11. The method according to claim 1, wherein the catalytic nucleic acid molecule is DNAzyme or RNAzyme.

12. The method according to claim 1, wherein the specimen is milk or a milk product, the target is melamine, and a filler of the column chromatography is a cation exchange resin.

13. A method for analyzing a target comprising:
bringing the sample produced by the method according to claim 1 into contact with a first binding substance that binds to a target and a catalytic nucleic acid molecule that generates a catalytic function to form a complex of the target in the sample, the first binding substance, and the catalytic nucleic acid molecule; and
detecting the catalytic function of the catalytic nucleic acid molecule in the complex to detect the target in the sample.

14. The method according to claim 13, wherein the first binding substance is a binding nucleic acid molecule that binds to the target.

15. The method according to claim 13, wherein the first binding substance is an antibody that binds to the target.

16. The method according to claim 13, wherein the complex forming step is a step of bringing an analysis element in which the first binding substance and the catalytic nucleic acid molecule are linked into contact with the sample.

17. The method according to claim 13, wherein the complex forming step is a step of bringing the first binding substance and a second binding substance that is modified with the catalytic nucleic acid molecule and binds to the first binding substance separately into contact with the sample.

18. The method according to claim 17, wherein the second binding substance is a binding nucleic acid molecule that binds to the first binding substance.

19. The method according to claim 17, wherein the second binding substance is an antibody that binds to the first binding substance.
